SEQUENCE LISTING (4 sequences)

SEQ ID NO: 1 - DNA, 30 bp, Artificial Sequence
  Description of Artificial Sequence Synthetic primer
atagaattcatgaatcagaatctgctggtg

SEQ ID NO: 2 - DNA, 30 bp, Artificial Sequence
  Description of Artificial Sequence Synthetic primer
atatctagatcagagctggaagttactcaa

SEQ ID NO: 3 - protein, 761 aa, Escherichia coli
MNQNLLVTKRDGSTERINLDKIHRVLDWAAEGLHNVSISQVELRSHIQFYDGIKTSDIHETIIKAAADLISRDAPDYQYLAARLAIFHLRKKAYGQFEPPALYDHVVKMVEMGKYDNHLLEDYTEEEFKQMDTFIDHDRDMTFSYAAVKQLEGKYLVQNRVTGEIYESAQFLYILVAACLFSNYPRETRLQYVKRFYDAVSTFKISLPTPIMSGVRTPTRQFSSCVLIECGDSLDSINATSSAIVKYVSQRAGIGINAGRIRALGSPIRGGEAFHTGCIPFYKHFQTAVKSCSQGGVRGGAATLFYPMWHLEVESLLVLKNNRGVEGNRVRHMDYGVQINKLMYTRLLKGEDITLFSPSDVPGLYDAFFADQEEFERLYTKYEKDDSIRKQRVKAVELFSLMMQERASTGRIYIQNVDHCNTHSPFDPAIAPVRQSNLCLEIALPTKPLNDVNDENGEIALCTLSAFNLGAINNLDELEELAILAVRALDALLDYQDYPIPAAKRGAMGRRTLGIGVINFAYYLAKHGKRYSDGSANNLTHKTFEAIQYYLLKASNELAKEQGACPWFNETTYAKGILPIDTYKKDLDTIANEPLHYDWEALRESIKTHGLRNSTLSALMPSETSSQISNATNGIEPPRGYVSIKASKDGILRQVVPDYEHLHDAYELLWEMPGNDGYLQLVGIMQKFIDQSISANTNYDPSRFPSGKVPMQQLLKDLLTAYKFGVKTLYYQNTRDGAEDAQDDLVPSIQDDGCESGACKI

SEQ ID NO: 4 - protein, 376 aa, Escherichia coli
MAYTTFSQTKNDQLKEPMFFGQPVNVARYDQQKYDIFEKLIEKQLSFFWRPEEVDVSRDRIDYQALPEHEKHIFISNLKYQTLLDSIQGRSPNVALLPLISIPELETWVETWAFSETIHSRSYTHIIRNIVNDPSVVFDDIVTNEQIQKRAEGISSYYDELIEMTSYWHLLGEGTHTVNGKTVTVSLRELKKKLYLCLMSVNALEAIRFYVSFACSFAFAERELMEGNAKIIRLIARDEALHLTGTQHMLNLLRSGADDPEMAEIAEECKQECYDLFVQAAQQEKDWADYLFRDGSMIGLNKDILCQYVEYITNIRMQAVGLDLPFQTRSNPIPWINTWLVSDNVQVAPQEVEVSSYLVGQIDSEVDTDDLSNFQL

(19) United States
(12) Patent Application Publication
(10) Pub. No.: US 2004/0214291 A1
(43) Pub. Date: Oct. 28, 2004

(54) Deoxyribonucleotides manufacturing by enzymatic reduction of ribonucleotides

(75) Inventor: Kuo-Ming Yu, Taipei (TW)

Correspondence Address: Kent H. Cheng, Esq., Cohen, Pontani, Lieberman & Pavane, Suite 1210, 551 Fifth Avenue, New York, NY, 10176, US

(73) Assignee: ScinoPharm Biotech, Ltd.

(21) Appl. No.: 10/746,396

(22) Filed: Dec. 23, 2003

Related U.S. Application Data
(60) Provisional application No. 60/436,282, filed on Dec. 23, 2002.

(51) Int. Cl. C12P 19/34; C12N 1/18

(52) U.S. Cl. 435/91.1; 435/254.2

(57) ABSTRACT

A method for in vitro preparation of deoxyribonucleotides is disclosed. The deoxyribonucleotides in the present invention are converted from ribonucleotides extracted from yeast in the presence of E. coli RNA reductase and a reducing agent.

### RELATED APPLICATIONS

This application claims priority from U.S. Provisional Patent Application Ser. No. 60/436,282 which was filed on Dec. 23, 2002, the content of which is hereby incorporated by reference in its entirety.

### BACKGROUND OF THE INVENTION

1. Field of the Invention

The present invention is directed to a method of preparing deoxyribonucleotides by enzymatic reduction of ribonucleotides extracted from yeast.

2. Description of the Related Art

Current commercial preparations of deoxyribonucleotides are extracted from salmon testes. They are expensive and generally in short supply. With increasing demand in deoxyribonucleotides as starting materials for synthetic “antisense” oligonucleotides for potential cancer treatment, an alternative source of deoxyribonucleotides will have to be sought. The technology described herein employs an enzymatic conversion process to obtain deoxyribonucleotides from their corresponding oxy-forms which are readily available from yeast, a source of abundant supply at low costs. The production of ribonucleotides is a mature skill and has been reported (Kuninaka, et al, Agric. Biol. Chem., 44, pp1821-27,1980). The phosphorylation of ribonucleotides has also been published for four decades (Laufer, et al, U.S. Pat. No. 3,138,539).

### SUMMARY OF THE INVENTION

An object of the present invention is to provide a method of preparing deoxyribonucleotides in vitro, which comprises the steps of:

a) preparing ribonucleotides extracted from yeast RNA;

b) phosphorylating said yeast ribonucleotides to produce a phosphorylated ribonucleotide product; and

c) converting said phosphorylated ribonucleotide product to deoxyribonucleotide product in a reaction solution containing a reducing agent and E. coli ribonucleotide reductase (RNR).

Another object of the present invention is to provide a deoxyribonucleotide product that is made by a process comprising the steps of:

c) preparing ribonucleotides extracted from yeast RNA;

d) phosphorylating said yeast ribonucleotides to produce a phosphorylated ribonucleotide product; and

c) converting said phosphorylated ribonucleotide product to deoxyribonucleotide product in a reaction solution containing a reducing agent and E. coli ribonucleotide reductase (RNR).

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIG. 1 shows the reaction product of dCDP analyzed by HPLC.

FIG. 2 shows the identity of reaction product (dCDP) by LC-MS.

FIG. 3 shows the reaction product of dGDP analyzed by HPLC.

FIG. 4 shows the identity of reaction product (dGDP) by LC-MS.

FIG. 5 shows the identity of dUDP by LC-MS.

FIG. 6 shows the product mixture analyzed by HPLC.

FIG. 7 shows the identity of reaction product (dAMP) by LC-MS.

FIG. 8 shows the product mixture as in FIG. 6 further treated with alkaline phosphatase to verify the identity of product.

FIG. 9 shows the example of consecutive reaction in an ultrafilter tube.

FIG. 10 shows the SDS-PAGE of RNR after Dialysis, (demonstrated by example 3).

### DETAILED DESCRIPTION OF THE PRESENTLY PREFERRED EMBODIMENTS

Abbreviations:

DTT: Dithiothreit

DNA: Deoxyribonucleic acids

NADPH: Nicotinamide adenine dinucleotide phosphate, reduced form

ADP: Adenosine 5′-diphosphate

UDP: Uridine 5′-diphosphate

GDP: Guanosine 5′-diphosphate

CDP: Cytidine 5′-diphosphate

AMP: Adenosine 5′-monophosphate

ATP: Adenosine 5′-triphosphate

dADP: Deoxyadenosine 5′-diphosphate

dUDP: Deoxyuridine 5′-diphosphate

dGDP: Deoxyguanosine 5′-diphosphate

dCDP: Deoxycytidine 5′-diphosphate

dAMP: Deoxyadenosine 5′-monophosphate

LC-MS: Liquid chromatography-MASS

IPTG: Isopropyl-beta-D-thiogalactopyranoside

HPLC: High performance liquid chromatography

The invention may be summarized schematically as follows:

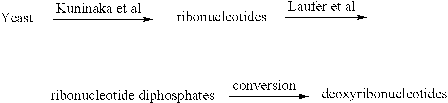

Ribonucleotide reductase (RNR) is the primary enzyme catalyzing the conversion reaction of ribonucleotides to deoxyriboinucleotides for in vivo DNA synthesis. RNR utilizes NADPH as the reducing agent and recycles it in vivo. The use of artificial agent DTT is not novel, since it has been used to measure RNR's activity in research laboratories. RNR is often over-expressed in tumor cells and drugs against RNR's activity were thus assayed. Such a method required purified RNR from tissue and radioactive substrates to increase sensitivity.

The approach in the present invention is designed to manufacture deoxyribonucleotides in vitro. The purified RNR is not required in the present process and DTT may be replaced by a more economic reducing agent: beta-mercaptoethanol. An inexpensive substrate ribonucleotides may be obtained from yeast. The deoxyribonucleotide product may be separated from the enzymes by ultra-filtration. Furthermore, RNR is capable of recycling for consecutive reactions.

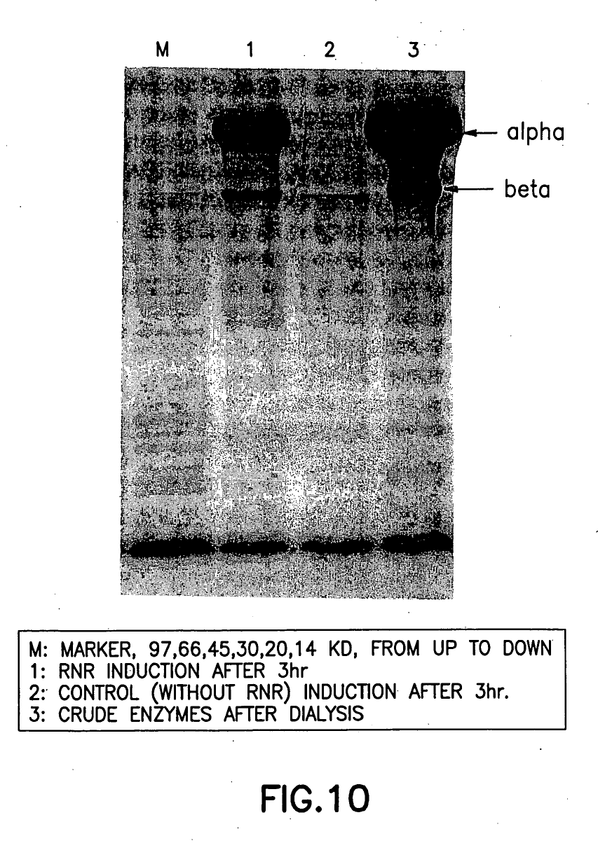

Although a pure or highly purified RNR is equally effective in the present invention, only partially purified RNR is needed in the present invention. The purity of the β-subunit for such partially purified RNR was shown as FIG. 10. The RNR may be partially purified by any method known to a person of ordinary skilled in the art.

In the class I reductase from E. coli, the enzyme, an α2β2 holoenzyme, consists of two homodimers, R1(M.W. 171 KD, 2×761 residues) and R2 (M.W. 87 KD, 2×375 residues). The R1 protein contains an active site and two allosteric binding sites; the R2 protein, on the other hand, contains a radical tyrosine side chain close to a binuclear iron center. Neither R1 nor R2 exhibits catalytic activities alone. The activity is initiated by the reduction of RNR with at least two reducing systems in vivo, i.e., thioredoxin and glutaredoxin. Both use NADPH as the ultimate reductant. Artificial reducing agents such as DTT or glutathione are as effective in vitro in our test.

The conversion process being developed involves cloning of α and β genes of RNR into an expression vector in tandem. The expressed enzyme, in intracellular, soluble form, was partially purified for the catalytic conversion of ribonucleotides to deoxyribonucleotides. The reducing agent, such as DTT has been found equally effective as the naturally occurring reducing agents such as thioredoxin or glutaredoxin. The scheme using recombinant RNR enzyme with addition of the reducing agent such as DTT is capable of converting ribonucleotides to deoxyribonucleotides in a commercial scale at reasonable costs, in contrast to the source currently available from salmon testes. Our results thus far indicate that the production of deoxy-nucleotides using RNR enzyme extract is feasible for substrate ADP, UDP, GDP and CDP. Unlike CDP, UDP and GDP being converted to their corresponding deoxy-diphosphate forms, ADP was converted to dAMP and the time consumed is prolonged. Thermal stability test also demonstrated that such an enzyme extract system is suitable for repetitive production. The system is designed as a membrane-like filter. Substrates could go through the membrane and the enzyme will be restrained in the membrane. Hence, the enzyme could be used repeatedly.

The following examples serve to illustrate the present invention, which should not be construed as a limitation of the scope of the claims.

### EXAMPLE 1

### Expression of RNR

The transformed E. coli BL21 strain with the plasmid containing RNR genes grew in LB broth at 37° C. Induction was initiated once OD600 reached at about 0.6. Incubation continued at 37° C. for another 3 hours after induction. Both α and β subunits were successfully expressed in soluble form.

Cloning of E. coli nrdAB Genes

E. coli RNR gene sequence is readily available in gene bank. Since E. coli nrdAB genes (coding for RNR alpha and beta subunits) are in a tandem, they were cloned by performing the polymerase chain reaction (PCR) with isolated E. coli genomic DNA. The primers for PCR nrdAB genes were:

| | |
|---|---|
| 5′-ATAGAATTCATGAATCAGAATCTGCTGGTG | (SEQ. ID. NO. 1) |
| 5′-ATATCTAGATCAGAGCTGGAAGTTACTCAA. | (SEQ. ID. NO. 2) |

The restriction site EcoRI was introduced at the beginning of the nrdAB, and XbaI was at the 3′ end of nrdAB.

The gene product of nrdA: (SEQ. ID. NO. 3)

MNQNLLVTKRDGSTERINLDKIHRVLDWAAEGLHNVSISQVELRSHIQFY
DGIKTSDIHETIIKAAADLISRDAPDYQYLAARLAIFHLRKKAYGQFEPP
ALYDHVVKMVEMGKYDNHLLEDYTEEEFKQMDTFIDHDRDMTFSYAAVKQ
LEGKYLVQNRVTGEIYESAQFLYILVAACLFSNYPRETRLQYVKRFYDAV
STFKISLPTPIMSGVRTPTRQFSSCVLIECGDSLDSINATSSAIVKYVSQ
RAGIGINAGRIRALGSPIRGGEAFHTGCIPFYKHFQTAVKSCSQGGVRGG
AATLFYPMWHLEVESLLVLKNNRGVEGNRVRHMDYGVQINKLMYTRLLKG
EDITLFSPSDVPGLYDAFFADQEEFERLYTKYEKDDSIRKQRVKAVELFS
LMMQERASTGRIYIQNVDHCNTHSPFDPAIAPVRQSNLCLEIALPTKPLN
DVNDENGEIALCTLSAFNLGAINNLDELEELAILAVRALDALLDYQDYPI
PAAKRGAMGRRTLGIGVINFAYYLAKHGKRYSDGSANNLTHKTFEAIQYY
LLKASNELAKEQGACPWFNETTYAKGILPIDTYKKDLDTIANEPLHYDWE
ALRESIKTHGLRNSTLSALMPSETSSQISNATNGIEPPRGYVSIKASKDG
ILRQVVPDYEHLHDAYELLWEMPGNDGYLQLVGIMQKFIDQSISANTNYD
PSRFPSGKVPMQQLLKDLLTAYKFGVKTLYYQNTRDGAEDAQDDLVPSIQ
DDGCESGACKI;

The gene product of nrdB gene: (SEQ. ID. NO. 4)

MAYTTFSQTKNDQLKEPMFFGQPVNVARYDQQKYDIFEKLIEKQLSFFWR
PEEVDVSRDRIDYQALPEHEKHIFISNLKYQTLLDSIQGRSPNVALLPLI
SIPELETWVETWAFSETIHSRSYTHIIRNIVNDPSVVFDDIVTNEQIQKR
AEGISSYYDELIEMTSYWHLLGEGTHTVNGKTVTVSLRELKKKLYLCLMS
VNALEAIRFYVSFACSFAFAERELMEGNAKIIRLIARDEALHLTGTQHML
NLLRSGADDPEMAEIAEECKQECYDLFVQAAQQEKDWADYLFRDGSMIGL
NKDILCQYVEYITNIRMQAVGLDLPFQTRSNPIPWINTWLVSDNVQVAPQ
EVEVSSYLVGQIDSEVDTDDLSNFQL)

Construct of Plasmid

The cloned grxA and nrdAB genes were respectively incoporated into pGEM-T Easy Vector (purchase from Promega), followed by cloning into the pET-30a expression vector (purchased from Novagen). The diagram of the vector was thus illustrated as followed:

[image]

The glutaredoxin was cloned for a recycling reducing agent in live cells. However our scheme was proven not feasible in vivo and therefore an in vitro test was carried out with partial purified RNR. An artificial reducing agent, DTT or beta-mercaptoethanol—a cheaper reducing agent, was introduced and therefore glutaredoxin is no longer relevant to this project but is still kept in the expression vector.

### EXAMPLE 2

### Fermentation Using Transformed Cells

Instead of deoxyribonucleotides, an unidentified product (might be hypoxanthine) was observed by using a living whole-cell system. It is postulated that the substrate undergoes different metabolite pathways from the one originally engineered. ATPase, a transmembrane protein prevailingly present in membrane, as well as other cytoplasmic phosphatase and kinase, may severely interfere RNR's activity. The enzymatic conversion scheme was therefore, subsequently tested in a cell-free system.

### EXAMPLE 3

### Production and Partial Purification of RNR

The crude enzyme was prepared by streptomycin sulfate precipitation of endogenous nucleic acids, followed by ammonium sulfate precipitation of enzymes. These crude enzyme preparations were used for all subsequent catalytic conversion tests.

10 ml transformed E. coli BL21 overnight culture was introduced to 1L LB medium. Induction was made by adding IPTG when OD600 reached 0.4-0.7. Cells were harvest at about 3 hr of induction. Cells were spin-down by centrifuge and washed with 20 mM Tris pH7.5 buffer. Sonicator or homogenizer was applied to break down cells. The supernatant of cell lysate was added one-fifth volume of 15% streptomycin sulfate to precipitate endogenous nucleic acids. After centrifuge, the supernatant was further treated with 55% ammonium sulfate to precipitate enzymes. The enzymes were re-suspended with 1-2 mL of 20 mM Tris pH7.5 buffer. The partially purified enzymes were subject to dialysis against 20 mM Tris pH7.5. AFTER THE DIALYSIS, the purity of the RESULTING RNR was shown as FIG. 10.

### EXAMPLE 4

### Preparation of Ribonucleotides from Yeast RNA

Ribonucleictides from yeast RNA may be prepared, for example, according to Kuninaka, et al, Agric. Biol. Chem., 44, pp1821-27,1980, which is incorporated by reference in its entirety. Any other methods that are known to, and can be readily performed by without undue experimentation, a person of ordinary skill in the art can be used to prepare ribonucleotides from yeast RNA for the purpose of the present invention as well.

### EXAMPLE 5

### Phosphorylation of Ribonucleotides

Phosphorylation of ribonnucleictides obtained from yeast may be conducted according to U.S. Pat. No. 3,138,539, which incorporated by reference in its entirety. In addition, the ribonucleotides may also be phosphoylated by any other methods that can be readily performed by a person of ordinary skill in the art without undue experimentation.

### EXAMPLE 6

### Catalytic Reactions to Convert Ribonucleotides to Deoxyribonucleotides

Reactions were carried out at 37° C./Tris buffer at pH 7.5, with DTT, Mg²⁺, and the substrates CDP, UDP, GDP and ADP. Addition of ATP to the reaction solution also facilitates the product's phosphorylation. The reaction solution was made by the following composition: 10 ul partial purified enzymes; 0.6 ul of 1M MgSO₄; 4 ul of 100 mM DTT; 2 ul of 50 mM substrate (ADP, GDP, CDP or UDP); add 20 mM Tris pH7.5 buffer to 10 ul. The reaction temperature was at 37° C. Reaction duration: 1 hr for UDP, CDP and GDP; 4 hr for ADP. The reaction solution was added with 900 ul water after reaction and subject to HPLC analysis immediately.

The HPLC analysis conditions were as followed:

Column: Supelcosil LC-18, 25 cm×4.6 mm, 5 um.

Mobile phases:

(Cytosine) isocratic flow 1 ml/min of 10 mM potassium phosphate pH6.5.

(Guanine) isocratic flow 1 ml/min of 10% Methanol in 100 mM potassium phosphate pH6.5.

(Uracil) isocratic flow 1 ml/min of 10 mM potassium phosphate pH6.5.

(Adenine) flow 1 ml/min of 5% to 10% methanol in 100 mM potassium phosphate pH6.5 over 5 min, and additional 5 min of 10% methanol in 100 mM potassium phosphate pH6.5.

Detection: (Cytosine: 271 nm) (Guanine: 253 nm) (Uracil:260 nm) (Adenine:259 nm)

CDP->dCDP:

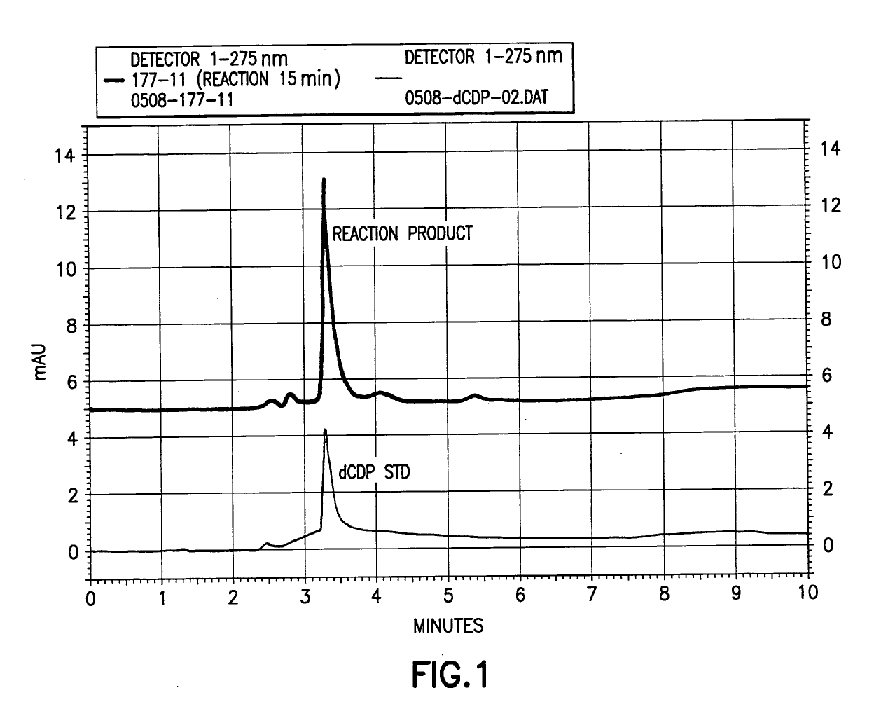

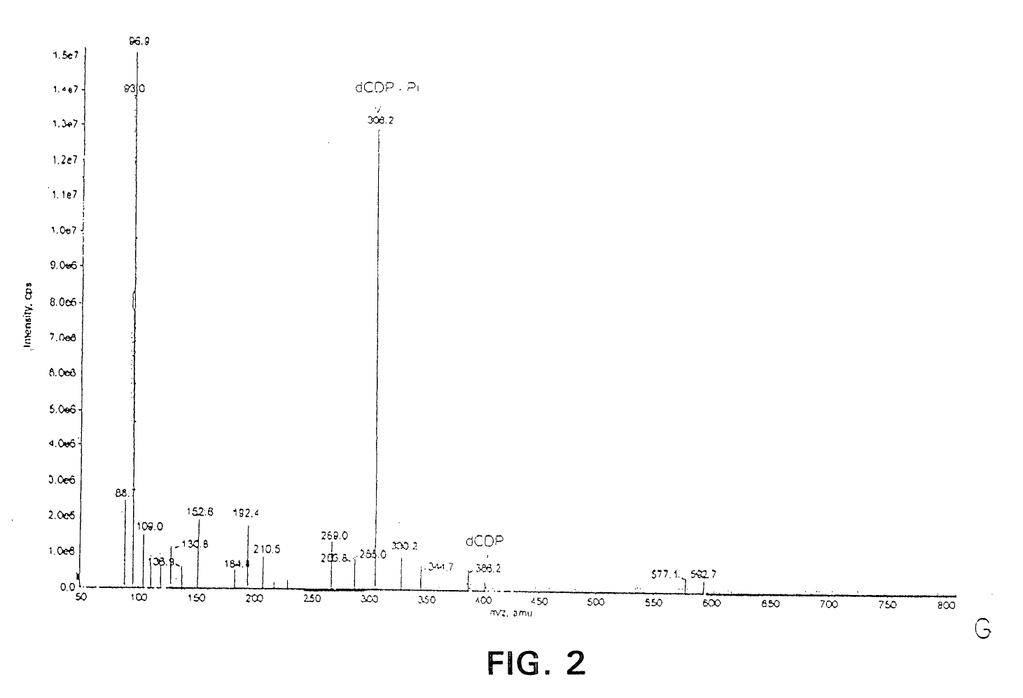

Nearly all CDP molecules were converted to dCDP as indicated in the following HPLC chromatogram. In the LC-MS diagram, fragments of dCDP (MW 387.2) and dCDP-Pi (307.2) were also identified. See FIG. 1 and FIG. 2.

GDP->dGDP:

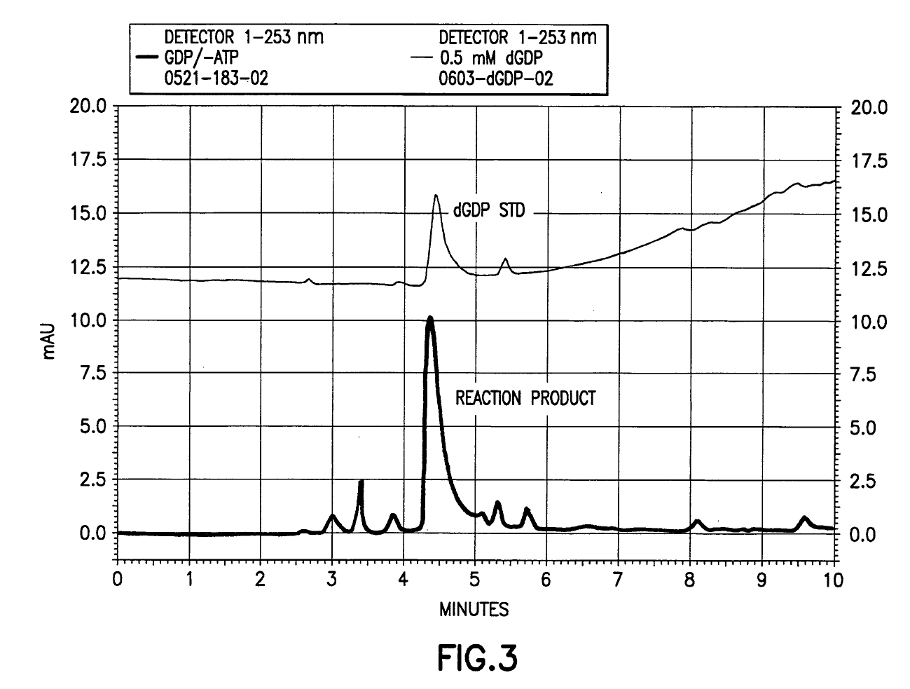

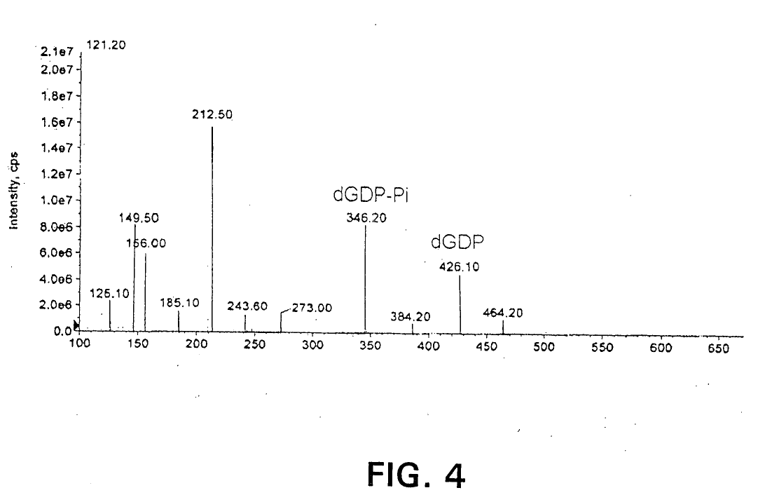

dGDP was produced as predominant species as indicated in the following HPLC chromatogram. In the LC-MS diagram, fragments of dGDP(MW 427.2) and dCDP-Pi (347.2) were also identified. See FIG. 3 and FIG. 4.

UDP->dUDP:

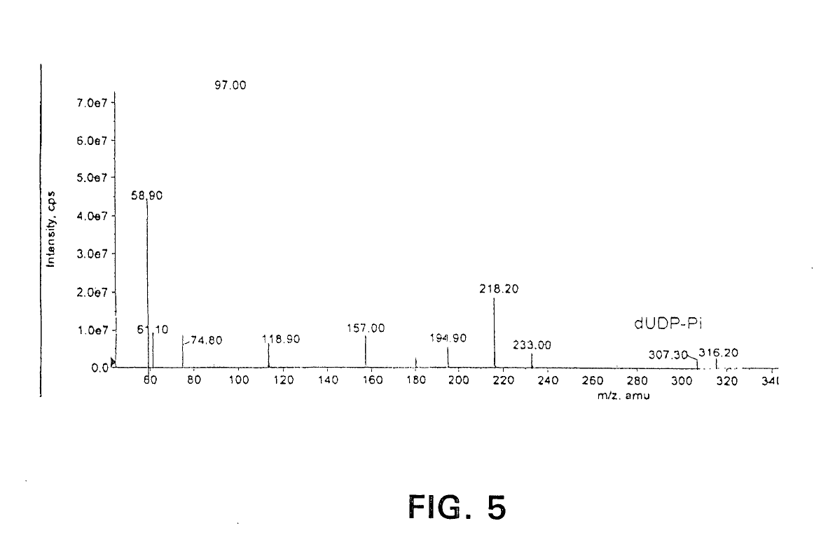

Due to the unavailability of standard dUDP, the reaction product was examined by LC-MS. The result was displayed in the following figure. Only fragment of dUDP-Pi (MW 388.2-80) was observed. See FIG. 5.

ADP->dADP->dAMP:

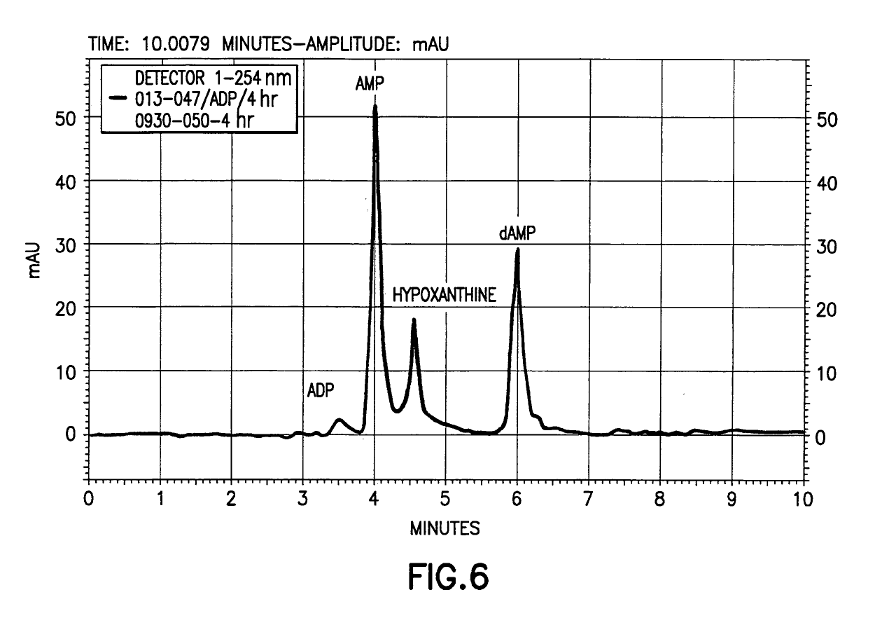

Unlike CDP/GDP/UDP being converted to their corresponding deoxy-diphosphates, ADP was converted to dADP, followed by further degradation (or catalyzed by endogenous phosphatase) to a more stable state—dAMP, after a 4-hour reaction. Three major products were identified: AMP, hypoxanthine and dAMP, with each content 49.1%, 16.7% and 31.7% respectively in an optimal case. See FIG. 6.

Besides examined by HPLC, the product dAMP was also verified by the following approaches:

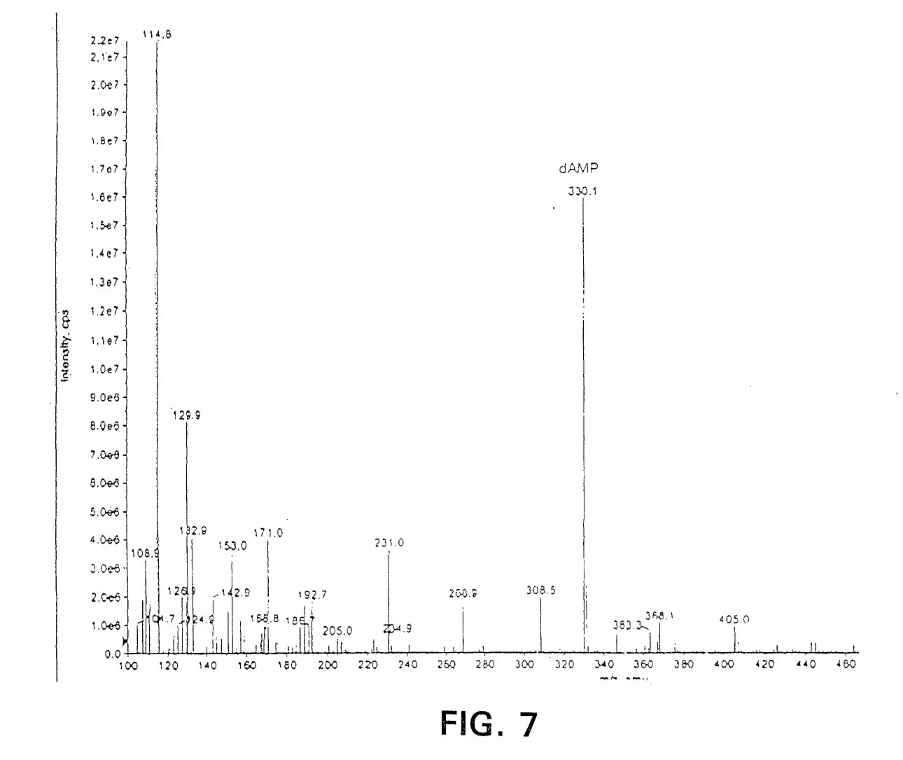

a) LC-MS: A peak corresponding to dAMP was obviously identified. See FIG. 7.

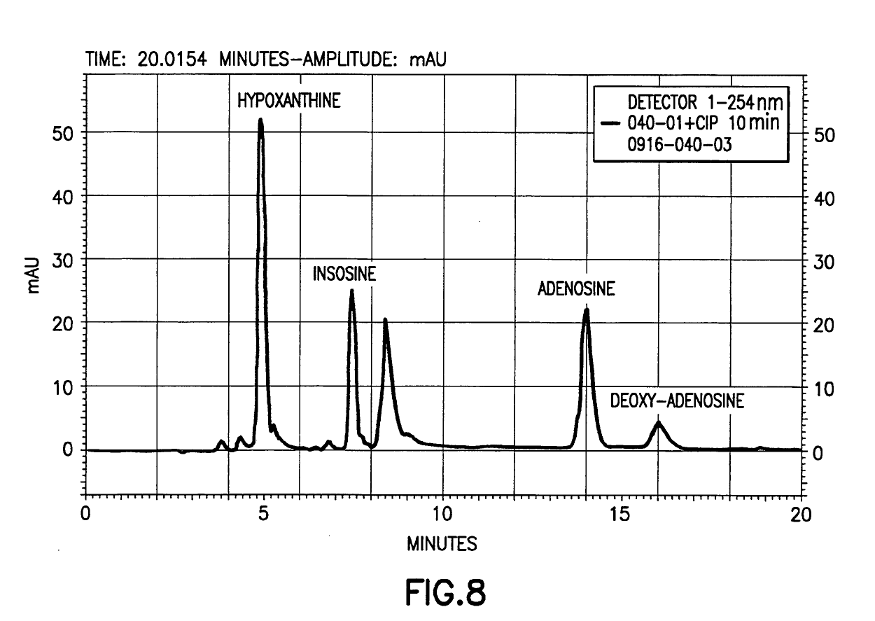

b) Treatment of phosphatase: The reaction products were treated with alkaline phosphatase, resulting in being further converted to adenosine, deoxy-adenosine, inosone and hypoxanthine as expected. Deoxy-adenosine was unstable and diminished soon over time. See FIG. 8.

### EXAMPLE 7

### Thermal Stability

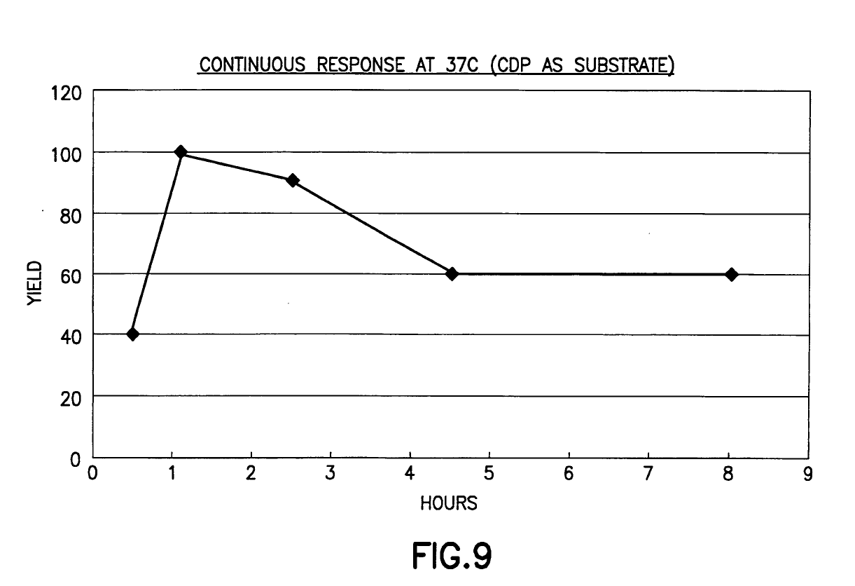

To demonstrate the concept of consecutive reactions in a small test-tube ultrafilter for conversion of CDP to dCDP, the crude enzyme was heated at 37° C. during the course of repetitive reactions. Results in the figure below indicate that, after five uses, the enzyme still retained approximately 60% of its maximum activity. The low activity of the initial reaction was probably due to precipitation of enzyme-substrate complex. See FIG. 9.

### EXAMPLE 8

### Immobilization of Crude Enzymes

For practical concerns, an attempt of immobilization of crude enzyme was carried out. The approach of using ion-exchange resin is not suitable because the substrates and the products are strong anions. The immobilization was thus carried out by entrapment with a couple of porous resins. All results failed to reveal catalytic activity of RNR, suggesting that with its size in about 300-400 Å, it is hard for RNR to penetrate the porous ranging from 200-600 Å. Coupled with the thermal stability result obtained above, it is therefore suggested that an ultra-filtration system might be suitable for separation of products from enzymes. See FIG. 10.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

I claim:

1. A method of preparing deoxyribonucleotides in vitro, comprising the steps of:
a) preparing ribonucleotides extracted from yeast RNA;
b) phosphorylating said yeast ribonucleotides to produce a phosphorylated ribonucleotide product; and
c) converting said phosphorylated ribonucleotide product to deoxyribonucleotide product.

2. The method of claim 1, wherein said step c) was performed in a reaction solution containing a reducing agent and E. coli ribonucleotide reductase (RNR).

3. The method of claim 2, wherein said E. coli rebonucleotide reductase (RNR) is partially purified.

4. A deoxyribonucleotide product prepared in vitro by a method comprising the steps of:
a) preparing ribonucleotides extracted from yeast RNA;
b) phosphorylating said yeast ribonucleotides to produce a phosphorylated ribonucleotide product; and
c) converting said phosphorylated ribonucleotide product to deoxyribonucleotide product.

5. The method of claim 4, wherein the step c) was performed in a reaction solution containing a reducing agent and E. coli ribonucleotide reductase (RNR).

6. The method of claim 5, wherein said E. coli rebonucleotide reductase (RNR) is partially purified.
